(19) United States
(12) Patent Application Publication
(10) Pub. No.: US 2017/0107173 A1
(43) Pub. Date: Apr. 20, 2017

(54) THERAPEUTIC COMPOUNDS AND METHODS

(71) Applicant: UNIVERSITY OF IOWA RESEARCH FOUNDATION, Iowa City, IA (US)

(72) Inventors: David F. Wiemer, Iowa City, IA (US); Jeffrey D. Neighbors, Iowa City, IA (US)

(21) Appl. No.: 15/293,840

(22) Filed: Oct. 14, 2016

Related U.S. Application Data
(60) Provisional application No. 62/242,185, filed on Oct. 15, 2015.

(51) Int. Cl. C07C 233/75; C07C 43/23

(52) U.S. Cl. CPC C07C 233/75; C07C 43/23

(57) ABSTRACT

Compounds of formula I:

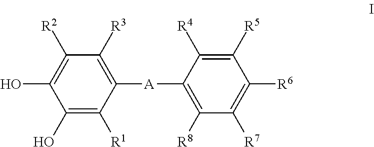

and salts thereof are disclosed. Also disclosed are pharmaceutical compositions comprising a compound of formula I, processes for preparing compounds of formula I, intermediates useful for preparing compounds of formula I and therapeutic methods for treating various diseases and conditions.

### RELATED APPLICATIONS

This application claims priority to U.S. Provisional Patent Application No. 62/242,185 filed Oct. 15, 2015, the entirety of which is incorporated herein by reference.

### GOVERNMENT FUNDING

This invention was made with government support under DA02-6573 awarded by The National Institutes of Health. The government has certain rights in the invention.

### BACKGROUND

The development of opioid compounds for treatment of pain is one of the triumphs of modern medicine (Pasternak, G. W. Clinical Neuropharmacology 1993, 16, 1). These compounds, however, are associated with numerous negative side effects, most prominently including sensitization to chronic treatment leading to development of addiction and the associated societal problems (Benyamin, R.; et al., Pain Physician 2008, 11, S105). The canonical opioid receptors kappa (KOP), mu (MOP), and delta (DOP) mediate a variety of key physiological processes, and are involved with the adaptation to chronic opioid analgesic treatment to different degrees (Pasternak, G. W. Clinical Neuropharmacology 1993, 16, 1, Raynor, K.; et al., Mol. Pharmacol. 1994, 45, 330). The primary analgesic response is attributable to activation of the MOP (Matthes, H. W. D.; et al., Nature 1996, 383, 819). The DOP is much less well studied but appears to play an interesting role in the development of learned habitual responses to chronic treatment with these potent analgesics (Gendron, L.; et al., J. Pharmacol. 2015, 172, 403). Because of this role in the addictive effects of the opioid pain medications, selective DOP receptor antagonists are gaining interest in the field of pain management and psychiatry (Abdelhamid, E. E.; et al., Journal of Pharmacology and Experimental Therapeutics 1991, 258, 299, Burford, N. T.; et al., J. Med. Chem. 2015, 58, 4220, Nemoto, T.; et al., Bioorg Med Chem Lett 2015, 25, 2927).

Isolation of the pawhuskin family of natural products has been reported (Belofsky, G.; et al., J. Nat. Prod. 2004, 67, 26). These compounds are non-nitrogenous opioid receptor modulators based around a stilbene core, and show significant potential as a scaffold for further exploration aimed at developing novel drug leads. There are several other non-nitrogenous scaffolds that are being studied as leads for opioid receptor modulators with the most prominent being the salvinorins, which have been studied predominantly as KOP agonists (Prisinzano, T. E. J. Med. Chem. 2013, 56, 3435, Riley, A. P.; et al., J. Med. Chem. 2014, 57, 10464, Simonson, B.; et al., Br. J. Pharmacol. 2015, 172, 515). Studies of the pawhuskins have led to the synthesis of pawhuskin A (1) (Neighbors, J. D.; et al., J. Nat. Prod. 2008, 71, 1949) and C (2) (Neighbors, J. D.; et al., Tetrahedron Lett. 2005, 46, 1321) (Scheme 1) as well as several analogues, and to the demonstration that compound 1 is a moderately selective KOP antagonist. For compound 3, the prenyl group on the “left-half” of the molecule (the portion biochemically derived from shikimate) is placed in a different orientation than in the parent pawhuskin A. This regioisomer turned out to be an opioid receptor antagonist with high selectivity for the KOP (δ/κ>67 and μ/κ>67) and slightly more potent than pawhuskin A (Kₑ=0.15 μM vs. 0.20 μM; Hartung, A. M.; et al., J. Nat. Prod. 2014, 77, 311).

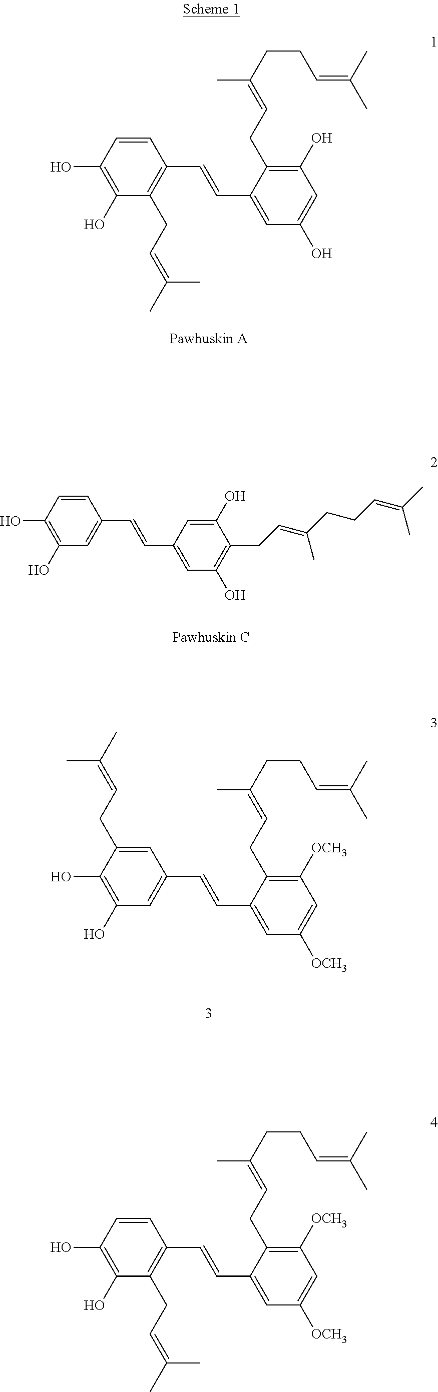 [SMILES: CC(C)=CCC/C(C)=C/Cc1c(O)cc(O)cc1/C=C/c1ccc(O)c(O)c1CC=C(C)C ; CC(C)=CCC/C(C)=C/Cc1c(O)cc(/C=C/c2ccc(O)c(O)c2)cc1O ; COc1cc(/C=C/c2cc(O)c(O)c(CC=C(C)C)c2)c(C/C=C(\C)CCC=C(C)C)c(OC)c1 ; COc1cc(/C=C/c2ccc(O)c(O)c2CC=C(C)C)c(C/C=C(\C)CCC=C(C)C)c(OC)c1]

There is currently a need for therapeutic agents and methods that modulate opioid receptors that are useful pain management or treating psychiatric conditions or substance dependency.

### SUMMARY OF THE INVENTION

Provided herein are compounds and methods that may be useful for the prevention or treatment of conditions or diseases such as but not limited to pain, psychiatric conditions or substance dependency.

Accordingly, one embodiment provides a compound of formula I:

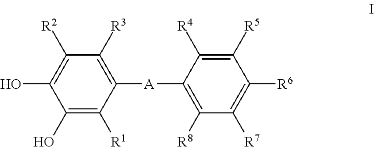

wherein;

A is

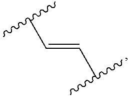 [SMILES: */C=C/*]

### —C(═O)NH— or —NHC(═O)—;

R¹ is a C₁-C₁₀ saturated or unsaturated hydrocarbon chain, aryl, heteroaryl, aryl(C₁-C₆)alkyl- or heteroaryl(C₁-C₆)alkyl- wherein any C₁-C₁₀ saturated or unsaturated hydrocarbon chain of R¹ is optionally substituted with one or more groups independently selected from halogen and —O(C₁-C₆)alkyl and any aryl, heteroaryl, aryl(C₁-C₆)alkyl- or heteroaryl(C₁-C₆)alkyl- of R¹ is optionally substituted with one more groups independently selected from halogen, (C₁-C₆)alkyl and —O(C₁-C₆)alkyl, and R² is hydrogen, halogen or —OH; or

R¹ is hydrogen, halogen or —OH, and R² is a C₁-C₁₀ saturated or unsaturated hydrocarbon chain, aryl, heteroaryl, aryl(C₁-C₆)alkyl- or heteroaryl(C₁-C₆)alkyl- wherein any C₁-C₁₀ saturated or unsaturated hydrocarbon chain of R² is optionally substituted with one or more groups independently selected from halogen and —O(C₁-C₆)alkyl and any aryl, heteroaryl, aryl(C₁-C₆)alkyl- or heteroaryl(C₁-C₆)alkyl- of R² is optionally substituted with one more groups independently selected from halogen, (C₁-C₆)alkyl and —O(C₁-C₆)alkyl, and R² is hydrogen, halogen or —OH;

R³ is hydrogen or halogen;

R⁴ is a C₃-C₁₄ saturated or unsaturated hydrocarbon chain, aryl, heteroaryl, aryl(C₁-C₆)alkyl- or heteroaryl(C₁-C₆)alkyl- wherein any C₃-C₁₄ saturated or unsaturated hydrocarbon chain of R⁴ is optionally substituted with one or more groups independently selected from halogen and —O(C₁-C₆)alkyl and any aryl, heteroaryl, aryl(C₁-C₆)alkyl- or heteroaryl(C₁-C₆)alkyl- of R⁴ is optionally substituted with one more groups independently selected from halogen, (C₁-C₆)alkyl and —O(C₁-C₆)alkyl;

R⁵ is (C₁-C₄)alkyl, (C₂-C₄)alkenyl, (C₂-C₄)alkynyl or —OR^{a} wherein any (C₁-C₄)alkyl, (C₂-C₄)alkenyl or (C₂-C₄)alkynyl of R⁵ is optionally substituted with one or more halogen;

R⁶ is hydrogen, halogen, (C₁-C₄)alkyl, (C₂-C₄)alkenyl, (C₂-C₄)alkynyl or —OR^{b} wherein any (C₁-C₄)alkyl, (C₂-C₄)alkenyl or (C₂-C₄)alkynyl of R⁵ is optionally substituted with one or more halogen;

R⁷ is (C₁-C₄)alkyl, (C₂-C₄)alkenyl, (C₂-C₄)alkynyl or —OR^{a} wherein any (C₁-C₄)alkyl, (C₂-C₄)alkenyl or (C₂-C₄)alkynyl of R⁵ is optionally substituted with one or more halogen;

R⁸ is hydrogen or halogen;

R^{a} is (C₁-C₄)alkyl, (C₂-C₄)alkonyl or (C₂-C₄)alkynyl, wherein any (C₁-C₄)alkyl, (C₂-C₄)alkenyl or (C₂-C₄)alkynyl of R^{a} is optionally substituted with one or more halogen;

R^{b} is (C₁-C₄)alkyl, (C₂-C₄)alkenyl or (C₂-C₄)alkynyl, wherein any (C₁-C₄)alkyl, (C₂-C₄)alkenyl or (C₂-C₄)alkynyl of R^{b} is optionally substituted with one or more halogen; and

R^{c} is a (C₁-C₄)alkyl, (C₂-C₄)alkenyl or (C₂-C₄)alkynyl, wherein any (C₁-C₄)alkyl, (C₂-C₄)alkenyl or (C₂-C₄)alkynyl of R^{c} is optionally substituted with one or more halogen;

or a salt thereof;

provided the compound is not 5-((E)-2-((E)-3,7-dimethylocta-2,6-dien-1-yl)-3,5-dimethoxystyryl)-3-(3-methylbut-2-en-1-yl)benzene-1,2-diol or a salt thereof.

One embodiment provides a pharmaceutical composition comprising a compound of formula I or a pharmaceutically acceptable salt thereof as described herein, and a pharmaceutically acceptable carrier.

One embodiment provides a method for treating pain in an animal (e.g., a mammal such as a human), comprising administering to the animal in need thereof a therapeutically effective amount of a compound of formula I or a pharmaceutically acceptable salt thereof as described herein.

One embodiment provides a method for treating a psychiatric condition in an animal (e.g., a mammal such as a human), comprising administering to the animal in need thereof a therapeutically effective amount of a compound of formula I or a pharmaceutically acceptable salt thereof as described herein.

One embodiment provides a method for treating substance dependency in an animal (e.g., a mammal such as a human), comprising administering to the animal in need thereof a therapeutically effective amount of a compound of formula I or a pharmaceutically acceptable salt thereof as described herein.

One embodiment provides a method for antagonizing a delta opioid receptor in a cell, comprising contacting the cell in vitro or in vivo with a compound of formula I or a pharmaceutically acceptable salt thereof as described herein.

One embodiment provides a compound of formula I or a pharmaceutically acceptable salt thereof as described herein for use in medical therapy.

One embodiment provides a compound of formula I or a pharmaceutically acceptable salt thereof as described herein for the prophylactic or therapeutic treatment of pain, psychiatric conditions or substance abuse.

One embodiment provides the use of a compound of formula I or a pharmaceutically acceptable salt thereof as described herein for the manufacture of a medicament for the treatment of pain, psychiatric conditions or substance abuse.

One embodiment provides novel processes and novel intermediates disclosed herein that are useful for preparing compounds of formula I or salts thereof,

### BRIEF DESCRIPTION OF THE FIGURES

FIG. 1 illustrates the antagonist activity of compound 4 at the DOP.

FIGS. 2A, 2B, 2C and 2D illustrate docking studies with compound 4. FIG. 2A shows the structure of compound 4 bound in the DOP looking down into the active site from the top shows the key proposed H-bonds by lines. FIG. 2B shows compound 4 (lighter shade) and naltrindole (darker shade, from the crystal structure) shown in the active site. FIG. 2C shows the lowest energy docking poses of both compound 3 (darker shade) and compound 4 (lighter shade). FIG. 2D shows key features of the pharmacophore of compound 4 based on the message and address concept of opioid pharmacology, with message region interactions by darker shaded line and address region interactions by lighter shaded line. The graphics were rendered using the Chimera software suite.

### DETAILED DESCRIPTION

### Definitions

The term “alkyl” as used herein refers to a saturated linear or branched-chain hydrocarbon radical. Such radicals are exemplified by, but not limited to methyl (Me, —CH₃), ethyl (Et, —CH₂CH₃), 1-propyl (n-Pr, n-propyl, —CH₂CH₂CH₃), 2-propyl (i-Pr, i-propyl, —CH(CH₃)₂), 1-butyl (n-Bu, n-butyl, —CH₂CH₂CH₂CH₃), 2-methyl-1-propyl (i-Bu, i-butyl, —CH₂CH(CH₃)₂), 2-butyl (s-Bu, s-butyl, —CH(CH₃)CH₂CH₃), 2-methyl-2-propyl (t-Bu, t-butyl, —C(CH₃)₃), 1-pentyl (n-pentyl, —CH₂CH₂CH₂CH₂CH₃), 2-pentyl (—CH(CH₃)CH₂CH₂CH₃), 3-pentyl (—CH(CH₂CH₃)₂), 2-methyl-2-butyl (—C(CH₃)₂CH₂CH₃), 3-methyl-2-butyl (—CH(CH₃)CH(CH₃)₂), 3-methyl-1-butyl (—CH₂CH₂CH(CH₃)₂), 2-methyl-1-butyl (—CH₂CH(CH₃)CH₂CH₃), 1-hexyl (—CH₂CH₂CH₂CH₂CH₂CH₃), 2-hexyl (—CH(CH₃)CH₂CH₂CH₂CH₃), 3-hexyl (—CH(CH₂CH₃)(CH₂CH₂CH₃)), 2-methyl-2-pentyl (—C(CH₃)₂CH₂CH₂CH₃), 3-methyl-2-pentyl (—CH(CH₃)CH(CH₃)CH₂CH₃), 4-methyl-2-pentyl (—CH(CH₃)CH₂CH(CH₃)₂), 3-methyl-3-pentyl (—C(CH₃)(CH₂CH₃)₂), 2-methyl-3-pentyl (—CH(CH₂CH₃)CH(CH₃)₂), 2,3-dimethyl-2-butyl (—C(CH₃)₂CH(CH₃)₂), 3,3-dimethyl-2-butyl (—CH(CH₃)C(CH₃)₃, heptyl, octyl, nonyl, decyl, undecyl and dodecyl. For example, the term “(C₁-C₆)alkyl” as used herein refers to alkyl groups having from 1 to 6 carbon atoms which are straight or branched. The term “(C₁-C₈)alkyl” as used herein refers to alkyl groups having from 1 to 8 carbon atoms which are straight or branched.

The terms “alkenyl” or “alkene” as used herein refers a linear or branched-chain hydrocarbon radical with at least one carbon-carbon double bond (i.e., one or more double bonds). Such radicals are exemplified by vinyl(ethen-1-yl), allyl, 1-propenyl, 1-methylethen-1-yl, 1-buten-1-yl, 2-buten-1-yl, 3-buten-1-yl, 1-methyl-1-propen-1-yl, 2-methyl-1-propen-1-yl, 1-methyl-2-propen-1-yl, 2-methyl-2-propen-1-yl and 1-methyl-2-propen-1-yl and the like. For example, the term “(C₂-C₆)alkenyl” as used herein refers to alkenyl groups having from 2 to 6 carbon atoms which are straight or branched. The term “(C₂-C₈)alkenyl” as used herein refers to alkenyl groups having from 2 to 8 carbon atoms which are straight or branched.

The term “alkynyl” or “alkyne” as used herein refers to a linear or branched hydrocarbon radical with at least one carbon-carbon triple bond (i.e., one or more double bonds). Such radicals are exemplified by, but not limited to ethyn-1-yl, propyn-1-yl, propyn-2-yl, 1-methylprop-2-yn-1-yl, butyn-1-yl, butyn-2-yl, butyn-3-yl and the like. For example, the term “(C₂-C₆)alkynyl” as used herein refers to alkynyl groups having from 2 to 6 carbon atoms which are straight or branched. The term “(C₂-C₈)alkynyl” as used herein refers to alkynyl groups having from 2 to 8 carbon atoms which are straight or branched.

The term “halogen” or “halo” as used herein refers to fluoro, chloro, bromo and iodo.

As used herein, the term “saturated hydrocarbon chain” refers to a straight or branched hydrocarbon group that is saturated. As used herein, the term “unsaturated hydrocarbon chain” refers to a straight or branched hydrocarbon group that has at least one site of unsaturation (e.g., at least one double bond or triple bond (one or more double or triple bonds)).

The term “aryl” as used herein refers to a single aromatic ring or a multiple condensed ring system wherein the ring atoms are carbon. For example, an aryl group can have 6 to 10 carbon atoms, or 6 to 12 carbon atoms. Aryl includes a phenyl radical. Aryl also includes multiple condensed ring systems (e.g., ring systems comprising 2 rings) having about 9 to 12 carbon atoms or 9 to 10 carbon atoms in which at least one ring is aromatic. Such multiple condensed ring systems may be optionally substituted with one or more (e.g., 1 or 2) oxo groups on any carbocycle portion of the multiple condensed ring system. It is to be understood that the point of attachment of a multiple condensed ring system, as defined above, can be at any position of the ring system including an aryl or a carbocycle portion of the ring. Typical aryl groups include, but are not limited to, phenyl, indenyl, naphthyl, 1, 2, 3, 4-tetrahydronaphthyl, anthracenyl, and the like.

The term “heteroaryl” as used herein refers to a single aromatic ring or a multiple condensed ring system. The term includes 5-6 membered single aromatic rings of from about 1 to 5 carbon atoms and about 1-4 heteroatoms selected from the group consisting of oxygen, nitrogen and sulfur in the rings. The sulfur and nitrogen atoms may also be present in an oxidized form provided the ring is aromatic. Such rings include but are not limited to pyridyl, pyrimidinyl, oxazolyl or furyl. The term also includes multiple condensed ring systems (e.g., ring systems comprising 2 rings) wherein a heteroaryl group, as defined above, can be condensed with another heteroaryl (e.g., 1,5-naphthyridinyl), a carbocycle (e.g., 5,6,7,8-tetrahydroquinolyl) or an aryl (e.g. indazolyl) to form a multiple condensed ring system. Such multiple condensed ring systems may be optionally substituted with one or more (e.g., 1 or 2) oxo groups on the carbocycle portion of the condensed ring. In one embodiment a monocyclic or bicyclic heteroaryl has 5 to 10 ring atoms comprising 1 to 9 carbon atoms and 1 to 5 heteroatoms. It is to be understood that the point of attachment of a multiple condensed ring system (as defined above for a heteroaryl) can be at any position of the multiple condensed ring system including a heteroaryl, heterocycle, aryl or carbocycle portion of the multiple condensed ring system and at any suitable atom of the multiple condensed ring system including a carbon atom and heteroatom (e.g., a nitrogen). Exemplary heteroaryls include but are not limited to pyridyl, pyrrolyl, pyrazinyl, pyrimidinyl, pyridazinyl, pyrazolyl, thienyl, indolyl, imidazolyl, oxazolyl, thiazolyl, furyl, oxadiazolyl, thiadiazolyl, quinolyl, isoquinolyl, benzothiazolyl, benzoxazolyl, indazolyl, quinoxalyl, quinazolyl, 5,6,7,8-tetrahydroisoquinolinyl, benzofuranyl, benzimidazolyl and thianaphthenyl.

The term “carbocyclyl” or “carbocycle” as used herein refers to a saturated or partially unsaturated carbocyclic ring system wherein all the ring atoms are carbon. In one embodiment the carbocycle is a monocyclic or bicyclic of 3 to 12 carbon atoms in the ring. One such carbocycle is a “(C₃-C₈)carbocycle”.

The term “treat”, “treatment” or “treating,” to the extent it relates to a disease or condition includes preventing the disease or condition from occurring, inhibiting the disease or condition, eliminating the disease or condition, and/or relieving one or more symptoms of the disease or condition. The term “patient” as used herein refers to any animal including mammals such as humans, higher non-human primates, rodents domestic and farm animals such as cow, horses, dogs and cats. In one embodiment, the patient is a human patient. The phrase “therapeutically effective amount” means an amount of a compound described herein that (i) treats or prevents the particular disease, condition, or disorder, (ii) attenuates, ameliorates, or eliminates one or more symptoms of the particular disease, condition, or disorder, or (iii) prevents or delays the onset of one or more symptoms of the particular disease, condition, or disorder described herein.

Methods involving contacting, a cell include contacting in vitro and in vivo (e.g., a cell in an animal such as a mammal including a human).

The compounds disclosed herein can also exist as tautomeric isomers in certain cases. Although only one delocalized resonance structure may be depicted, all such forms are contemplated within the scope of the invention.

It is understood by one skilled in the art that this invention also includes any compound claimed that may be enriched at any or all atoms above naturally occurring isotopic ratios with one or more isotopes such as, but not limited to, deuterium (²H or D). As a non-limiting example, a —CH₃ group may be substituted with —CD₃.

It will be appreciated by those skilled in the art that compounds of formula I having a chiral center may exist in and be isolated in optically active and racemic forms. For example, it is possible for one or both phosphorous atoms in a compound of formula I to be chiral centers.

Some compounds may exhibit polymorphism. It is to be understood that the compounds of formula I can encompasses any racemic, optically-active, polymorphic, or stereoisomeric form, or mixtures thereof, of a compound of the invention, which possess the useful properties described herein, it being well known in the art how to prepare optically active forms (for example, by resolution of the racemic form by recrystallization techniques, by synthesis from optically-active starting materials, by chiral synthesis, or by chromatographic separation using a chiral stationary phase) and how to determine enzyme inhibitory activity using the standard tests that are well known in the art.

When a bond in a compound formula herein is drawn in a non-stereochemical manner (e.g. flat), the atom to which the bond is attached includes all stereochemical possibilities. When a bond in a compound formula herein is drawn in a defined stereochemical manner (e.g. bold, bold-wedge, dashed or dashed-wedge), it is to be understood that the atom to which the stereochemical bond is attached is enriched in the absolute stereoisomer depicted unless otherwise noted. In one embodiment, a mixture comprising the compound is at least 51% the absolute stereoisomer depicted. In one embodiment, a mixture comprising the compound is at least 60% the absolute stereoisomer depicted. In one embodiment, a mixture comprising the compound is at least 80% the absolute stereoisomer depicted. In one embodiment, a mixture comprising the compound is at least 90% the absolute stereoisomer depicted. In one embodiment, a mixture comprising the compound is at least 95% the absolute stereoisomer depicted. In one embodiment, a mixture comprising the compound is at least 99% the absolute stereoisomer depicted.

Specific embodiments listed below for radicals, substituents, and ranges, are for illustration only. The specific embodiments listed include embodiments for compounds of formula I and all subformulas thereof (e.g., formula I′, I′a, Ia, Ib, Ic, Id, Ie) which compounds are useful in the methods of the invention. One or more embodiments may be combined. It is also to be understood that one or more embodiments alone or in combination may be excluded.

One embodiment provides a compound of formula I″:

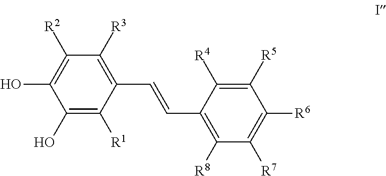

wherein;

R¹ is a C₁-C₁₀ saturated or unsaturated hydrocarbon chain, aryl, heteroaryl, aryl(C₁-C₆)alkyl- or heteroaryl(C₁-C₆)alkyl- wherein any C₁-C₁₀ saturated or unsaturated hydrocarbon chain of R¹ is optionally substituted with one or more (e.g., 1, 2, 3, 4 or 5) groups independently selected from halogen and —O(C₁-C₆)alkyl and any aryl, heteroaryl, aryl(C₁-C₆)alkyl- or heteroaryl(C₁-C₆)alkyl- of R¹ is optionally substituted with one more (e.g., 1, 2, 3, 4 or 5) groups independently selected from halogen, (C₁-C₆)alkyl and —O(C₁-C₆)alkyl;

R² is hydrogen, halogen or —OH;

R³ is hydrogen or halogen;

R⁴ is a C₃-C₁₄ saturated or unsaturated hydrocarbon chain, aryl, heteroaryl, aryl(C₁-C₆)alkyl- or heteroaryl(C₁-C₆)alkyl- wherein any C₃-C₁₄ saturated or unsaturated hydrocarbon chain of R⁴ is optionally substituted with one or more (e.g., 1, 2, 3, 4 or 5) groups independently selected from halogen and —O(C₁-C₆)alkyl and any aryl, heteroaryl, aryl(C₁-C₆)alkyl- or heteroaryl(C₁-C₆)alkyl- of R⁴ is optionally substituted with one more (e.g., 1, 2, 3, 4 or 5) groups independently selected from halogen, (C₁-C₆)alkyl and —O(C₁-C₆)alkyl;

R⁵ is a (C₁-C₄)alkyl, (C₂-C₄)alkenyl, (C₂-C₄)alkynyl or —OR^{a} wherein any (C₁-C₄)alkyl, (C2-C4)alkenyl or (C₂-C₄)alkynyl of R⁵ is optionally substituted with one or more (e.g., 1, 2, 3, 4 or 5) halogen;

R⁶ is hydrogen, halogen, (C₁-C₄)alkyl, (C₂-C₄)alkenyl, (C₂-C₄)alkynyl or —OR^{b} wherein any (C₁-C₄)alkyl, (C₂-C₄)alkenyl or (C₂-C₄)alkynyl of R⁵ is optionally substituted with one or more halogen;

R⁷ is (C₁-C₄)alkyl, (C₂-C₄)alkenyl, (C₂-C₄)alkynyl or —OR^{c} wherein any (C₁-C₄)alkyl, (C₂-C₄)alkenyl or (C₂-C₄)alkynyl of R⁵ is optionally substituted with one or more (e.g., 1, 2, 3, 4 or 5) halogen;

R⁸ is hydrogen or halogen;

R^{a} is (C₁-C₄)alkyl, (C₂-C₄)alkenyl or (C₂-C₄)alkynyl, wherein any (C₁-C₄)alkyl, (C₂-C₄)alkenyl or (C₂-C₄)alkynyl of R^{a} is optionally substituted with one or more (e.g., 1, 2, 3, 4 or 5) halogen;

R^{b} is (C₁-C₄)alkyl, (C₂-C₄)alkenyl or (C₂-C₄)alkynyl, wherein any (C₁-C₄)alkyl, (C₂-C₄)alkenyl or (C₂-C₄)alkynyl of R^{b} is optionally substituted with one or more (e.g., 1, 2, 3, 4 or 5) halogen; and

R^{c} is (C₁-C₄)alkyl, (C₂-C₄)alkenyl or (C₂-C₄)alkynyl, wherein any (C₁-C₄)alkyl, (C₂-C₄)alkenyl or (C₂-C₄)alkynyl of R^{c} is optionally substituted with one or more (e.g., 1, 2, 3, 4 or 5) halogen;

or a salt thereof.

In one embodiment R² is hydrogen.

In one embodiment R³ is hydrogen.

In one embodiment R⁶ is hydrogen.

In one embodiment R⁸ is hydrogen.

One embodiment provides a compound of formula I′a:

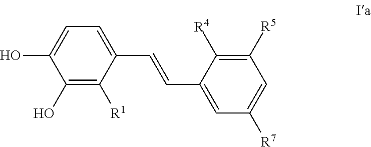

or a salt thereof.

In one embodiment R¹ is a C₃-C₇ saturated or unsaturated hydrocarbon chain wherein the hydrocarbon chain is optionally substituted with one or more halogen.

In one embodiment R¹ is a C₃-C₇ saturated or unsaturated hydrocarbon chain.

In one embodiment R¹ is a C₄-C₆ saturated or unsaturated hydrocarbon chain.

In one embodiment R¹ is:

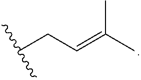 [SMILES: *CC=C(C)C]

In one embodiment R⁴ is a C₈-C₁₂ saturated or unsaturated hydrocarbon chain wherein the hydrocarbon chain is optionally substituted with one or more groups independently selected from halogen and —O(C₁-C₆)alkyl.

In one embodiment R⁴ is a C₈-C₁₂ saturated or unsaturated hydrocarbon chain wherein the hydrocarbon chain is optionally substituted with one or more halogen.

In one embodiment R⁴ is a C₈-C₁₂ saturated or unsaturated hydrocarbon chain.

In one embodiment R⁴ is a C₉-C₁₁ saturated or unsaturated hydrocarbon chain.

In one embodiment R⁴ is:

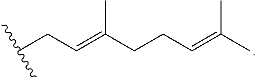 [SMILES: *C/C=C(\C)CCC=C(C)C]

In one embodiment R⁵ is —OR^{a}.

In one embodiment R^{a} is a (C₁-C₄)alkyl wherein any C₁-C₄)alkyl of R^{a} is optionally substituted with one or more halogen.

In one embodiment R⁵ is —OCH₃.

In one embodiment R⁷ is —OR^{c}.

In one embodiment R^{c} is a (C₁-C₄)alkyl wherein any (C₁-C₄)alkyl of R^{b} is optionally substituted with one or more halogen.

In one embodiment R⁷ is —OCH₃.

In one embodiment R⁶ is —OR^{b}.

In one embodiment R^{b} is a (C₁-C₄)alkyl wherein any (C₁-C₄)alkyl of R^{b} is optionally substituted with one or more halogen.

In one embodiment R⁶ is —OCH₃.

In one embodiment R^{a} and R^{c} are each independently (C₁-C₄)alkyl wherein any (C₁-C₄)alkyl of R^{a} or R^{c} is optionally substituted with one or more halogen.

One embodiment provides a compound of formula Ia:

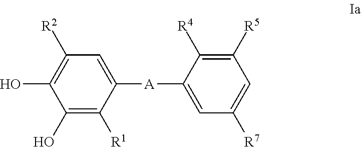

or a salt thereof.

One embodiment provides a compound of formula Ib:

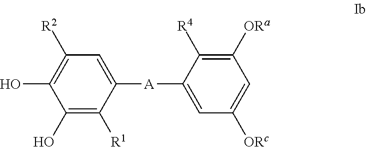

or a salt thereof.

One embodiment provides a compound of formula Ic, 1d, or 1e:

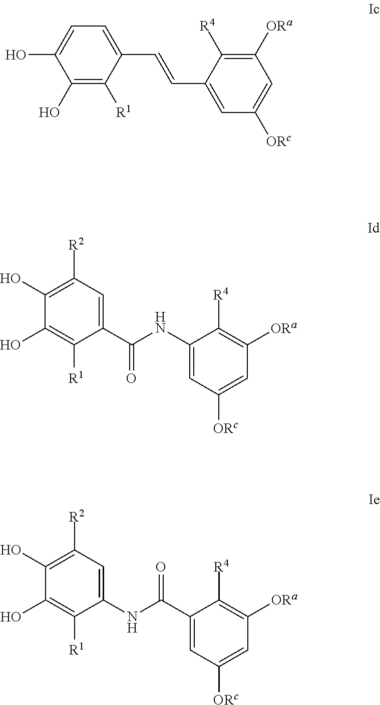

or a salt thereof.

In one embodiment R¹ is a C₃-C₇ saturated or unsaturated hydrocarbon chain wherein the hydrocarbon chain is optionally substituted with one or more groups independently selected from halogen and —O(C₁-C₆)alkyl and R² is hydrogen, halogen or —OH.

In one embodiment R² is a C₃-C₇ saturated or unsaturated hydrocarbon chain wherein the hydrocarbon chain is optionally substituted with one or more groups independently selected from halogen and —O(C₁-C₆)alkyl and R¹ is hydrogen, halogen or —OH.

In one embodiment R¹ is a C₃-C₇ saturated or unsaturated hydrocarbon chain wherein the hydrocarbon chain is optionally substituted with one or more groups independently selected from halogen and —O(C₁-C₆)alkyl, R² is hydrogen, halogen or —OH, and A is

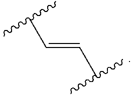 [SMILES: */C=C/*]

In one embodiment A is —C(═O)NH— or —NHC(═O)—, and R¹ is a C₃-C₇ saturated or unsaturated hydrocarbon chain wherein the hydrocarbon chain is optionally substituted with one or more groups independently selected from halogen and —O(C₁-C₆)alkyl and R² is hydrogen, halogen or —OH; or A is —C(═O)NH— or —NHC(═O)—, and R² is a C₃-C₇ saturated or unsaturated hydrocarbon chain wherein the hydrocarbon chain is optionally substituted with one or more groups independently selected from halogen and —O(C₁-C₆)alkyl and R¹ is hydrogen, halogen or —OH.

One embodiment provides the compound:

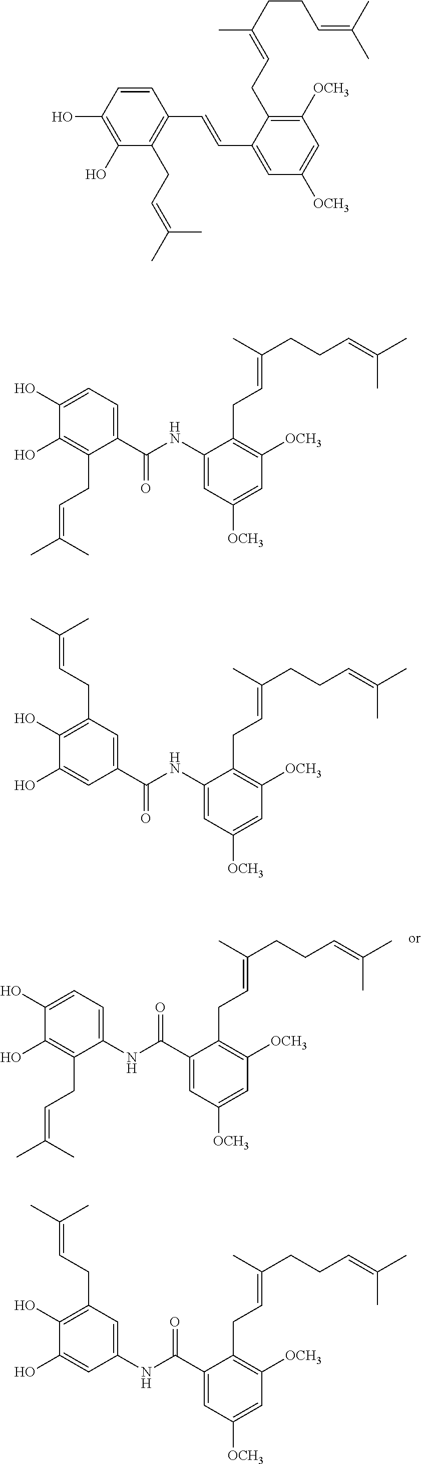 [SMILES: COc1cc(/C=C/c2ccc(O)c(O)c2CC=C(C)C)c(C/C=C(\C)CCC=C(C)C)c(OC)c1 ; COc1cc(NC(=O)c2ccc(O)c(O)c2CC=C(C)C)c(C/C=C(\C)CCC=C(C)C)c(OC)c1 ; COc1cc(NC(=O)c2cc(O)c(O)c(CC=C(C)C)c2)c(C/C=C(\C)CCC=C(C)C)c(OC)c1 ; COc1cc(OC)c(C/C=C(\C)CCC=C(C)C)c(C(=O)Nc2ccc(O)c(O)c2CC=C(C)C)c1 ; COc1cc(OC)c(C/C=C(\C)CCC=C(C)C)c(C(=O)Nc2cc(O)c(O)c(CC=C(C)C)c2)c1]

or a salt thereof.

One embodiment provides the compound:

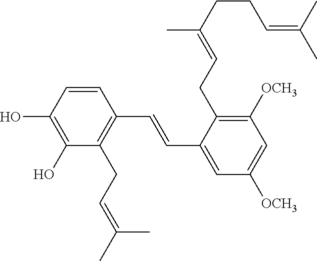 [SMILES: COc1cc(/C=C/c2ccc(O)c(O)c2CC=C(C)C)c(C/C=C(\C)CCC=C(C)C)c(OC)c1]

or a salt thereof.

In one embodiment the compound:

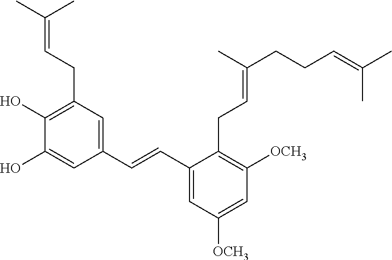 [SMILES: COc1cc(/C=C/c2cc(O)c(O)c(CC=C(C)C)c2)c(C/C=C(\C)CCC=C(C)C)c(OC)c1]

### 5-((E)-2-((E)-3,7-dimethylocta-2,6-dien-1-yl)-3,5-dimethoxystyryl)-3-(3-methylbut-2-en-1-yl)benzene-1,2-diol

or a salt thereof is excluded.

Processes for preparing compounds of formula I are provided as embodiments of the invention.

In cases where compounds are sufficiently basic or acidic to form stable nontoxic acid or base salts, administration of the compounds as salts may be appropriate. Examples of pharmaceutically acceptable salts are organic acid addition salts formed with acids that form a physiological acceptable anion, for example, tosylate, methanesulfonate, acetate, citrate, malonate, tartrate, succinate, benzoate, ascorbate, α-ketoglutarate, and α-glycerophosphate. Suitable inorganic salts may also be formed, including hydrochloride, sulfate, nitrate, bicarbonate, and carbonate salts.

Pharmaceutically acceptable salts may be obtained using standard procedures well known in the art, for example by reacting a sufficiently basic compound such as an amine with a suitable acid affording a physiologically acceptable anion. Alkali metal (for example, sodium, potassium or lithium) or alkaline earth metal (for example calcium) salts of carboxylic acids can also be made.

The compounds of formula I can be formulated as pharmaceutical compositions and administered to a mammalian host, such as a human patient in a variety of forms adapted to the chosen route of administration, i.e., orally or parenterally, by intravenous, intramuscular, topical or subcutaneous routes.

Thus, the present compounds may be systemically administered, e.g., orally, in combination with a pharmaceutically acceptable vehicle such as an inert diluent or an assimilable edible carrier. They may be enclosed in hard or soft shell gelatin capsules, may be compressed into tablets, or may be incorporated directly with the food of the patient's diet. For oral therapeutic administration, the active compound may be combined with one or more excipients and used in the form of ingestible tablets, buccal tablets, troches, capsules, elixirs, suspensions, syrups, wafers, and the like. Such compositions and preparations should contain at least 0.1% of active compound. The percentage of the compositions and preparations may, of course, be varied and may conveniently be between about 2 to about 60% of the weight of a given unit dosage form. The amount of active compound in such therapeutically useful compositions is such that an effective dosage level will be obtained.

The tablets, troches, pills, capsules, and the like may also contain the following: binders such as gum tragacanth, acacia, corn starch or gelatin; excipients such as dicalcium phosphate; a disintegrating agent such as corn starch, potato starch, alginic acid and the like; a lubricant such as magnesium stearate; and a sweetening agent such as sucrose, fructose, lactose or aspartame or a flavoring agent such as peppermint, oil of wintergreen, or cherry flavoring may be added. When the unit dosage form is a capsule, it may contain, in addition to materials of the above type, a liquid carrier, such as a vegetable oil or a polyethylene glycol. Various other materials may be present as coatings or to otherwise modify the physical form of the solid unit dosage form. For instance, tablets, pills, or capsules may be coated with gelatin, wax, shellac or sugar and the like. A syrup or elixir may contain the active compound, sucrose or fructose as a sweetening agent, methyl and propylparabens as preservatives, a dye and flavoring such as cherry or orange flavor. Of course, any material used in preparing any unit dosage form should be pharmaceutically acceptable and substantially non-toxic in the amounts employed. In addition, the active compound may be incorporated into sustained-release preparations and devices.

The active compound may also be administered intravenously or intraperitoneally by infusion or injection. Solutions of the active compound or its salts can be prepared in water, optionally mixed with a nontoxic surfactant. Dispersions can also be prepared in glycerol, liquid polyethylene glycols, triacetin, and mixtures thereof and in oils. Under ordinary conditions of storage and use, these preparations contain a preservative to prevent the growth of microorganisms.

The pharmaceutical dosage forms suitable for injection or infusion can include sterile aqueous solutions or dispersions or sterile powders comprising the active ingredients which are adapted for the extemporaneous preparation of sterile injectable or infusible solutions or dispersions, optionally encapsulated in liposomes. In all cases, the ultimate dosage form should be sterile, fluid and stable under the conditions of manufacture and storage. The liquid carrier or vehicle can be a solvent or liquid dispersion medium comprising, for example, water, ethanol, a polyol (for example, glycerol, propylene glycol, liquid polyethylene glycols, and the like), vegetable oils, nontoxic glyceryl esters, and suitable mixtures thereof. The proper fluidity can be maintained, for example, by the formation of liposomes, by the maintenance of the required particle size in the case of dispersions or by the use of surfactants. The prevention of the action of microorganisms can be brought about by various antibacterial and antifungal agents, for example, parabens, chlorobutanol, phenol, sorbic acid, thimerosal, and the like. In many cases, it will be preferable to include isotonic agents, for example, sugars, buffers or sodium chloride. Prolonged absorption of the injectable compositions can be brought about by the use in the compositions of agents delaying absorption, for example, aluminum monostearate and gelatin.

Sterile injectable solutions are prepared by incorporating the active compound in the required amount in the appropriate solvent with various of the other ingredients enumerated above, as required, followed by filter sterilization. In the case of sterile powders for the preparation of sterile injectable solutions, the preferred methods of preparation are vacuum drying and the freeze drying techniques, which yield a powder of the active ingredient plus any additional desired ingredient present in the previously sterile-filtered solutions.

For topical administration, the present compounds may be applied in pure form, i.e., when they are liquids. However, it will generally be desirable to administer them to the skin as compositions or formulations, in combination with a dermatologically acceptable carrier, which may be a solid or a liquid.

Useful solid carriers include finely divided solids such as talc, clay, microcrystalline cellulose, silica, alumina and the like. Useful liquid carriers include water, alcohols or glycols or water-alcohol/glycol blends, in which the present compounds can be dissolved or dispersed at effective levels, optionally with the aid of non-toxic surfactants. Adjuvants such as fragrances and additional antimicrobial agents can be added to optimize the properties for a given use. The resultant liquid compositions can be applied from absorbent pads, used to impregnate bandages and other dressings, or sprayed onto the affected area using pump-type or aerosol sprayers.

Thickeners such as synthetic polymers, fatty acids, fatty acid salts and esters, fatty alcohols, modified celluloses or modified mineral materials can also be employed with liquid carriers to form spreadable pastes, gels, ointments, soaps, and the like, for application directly to the skin of the user.

Examples of useful dermatological compositions which can be used to deliver the compounds of formula I to the skin are known to the art; for example, see Jacquet et al. (U.S. Pat. No. 4,608,392), Geria (U.S. Pat. No. 4,992,478), Smith et al. (U.S. Pat. No. 4,559,157) and Wortzman (U.S. Pat. No. 4,820,508).

The compound of formula I can also be administered by inhalation. Formulations suitable for intrapulmonary or nasal administration typically have a particle size for example in the range of 0.1 to 500 microns, such as 0.5, 1, 30, 35 etc., which is administered by rapid inhalation through the nasal passage or by inhalation through the mouth so as to reach the alveolar sacs. Suitable formulations include aqueous or oily solutions of the active ingredient. Formulations suitable for aerosol or dry powder administration may be prepared using conventional methods. The compound of Formula I can be formulated for aerosol delivery using a nebulizer, pressurized metered dose inhaler (pMDI), or dry powder inhaler (DPI). Non-limiting examples of nebulizers include atomizing, jet, ultrasonic, pressurized, vibrating porous plate, or equivalent nebulizers including those nebulizers utilizing adaptive aerosol delivery technology.

Useful dosages of the compounds of formula I can be determined by comparing their in vitro activity, and in vivo activity in animal models. Methods for the extrapolation of effective dosages in mice, and other animals, to humans are known to the art; for example, see U.S. Pat. No. 4,938,949.

The amount of the compound, or an active salt or derivative thereof, required for use in treatment will vary not only with the particular salt selected but also with the route of administration, the nature of the condition being treated and the age and condition of the patient and will be ultimately at the discretion of the attendant physician or clinician.

The desired dose may conveniently be presented in a single dose or as divided doses administered at appropriate intervals, for example, as two, three, four or more sub-doses per day. The sub-dose itself may be further divided, e.g., into a number of discrete loosely spaced administrations.

The delta opioid receptor (DOR) is implicated in emotional and behavioral responses, reward and addiction, as well as additional diseases (Chung, P. C. S. and B. L. Kieffer, Delta opioid receptors in brain function and diseases. Pharmacology & Therapeutics, 2013. 140(1): p. 112-120). In addition, DOR antagonists appear to have potential as therapeutics for the treatment of alcohol and nicotine abuse (Berrendero, F., et al., Influence of delta-Opioid Receptors in the Behavioral Effects of Nicotine. Neuropsychopharmacology, 2012. 37(10): p. 2332-2344, Nielsen, C. K., et al., A Novel Delta Opioid Receptor Antagonist, SoRI-9409, Produces a Selective and Long-Lasting Decrease in Ethanol Consumption in Heavy-Drinking Rats. Biological Psychiatry, 2008. 64(11): p. 974-981).

Accordingly, the compounds of formula I or pharmaceutically acceptable salts thereof as described herein may be useful for treating or preventing pain, psychiatric conditions (e.g., depression) or substance dependency, abuse or addiction (e.g., alcohol, nicotine, opioid, or stimulant (e.g., methamphetamine) dependency, abuse or addiction).

In one embodiment the compound of formula I or salt thereof is a selective antagonist for the delta opioid receptor.

In one embodiment the compound of formula I or salt thereof is an antagonist for the delta opioid receptor with greater than or equal to two-fold antagonist selectivity for the delta opioid receptor over the kappa opioid receptor or the mu opioid receptor.

In one embodiment the compound of formula I or salt thereof is an antagonist for the delta opioid receptor with greater than or equal to ten-fold antagonist selectivity for the delta opioid receptor over the kappa opioid receptor or the mu opioid receptor.

In one embodiment the compound of formula I or salt thereof is an antagonist for the delta opioid receptor with greater than or equal to hundred-fold antagonist selectivity for the delta opioid receptor over the kappa opioid receptor or the mu opioid receptor.

The invention will now be illustrated by the following non-limiting Examples.

### EXAMPLE 1

### Synthesis of Compounds

The synthesis of pawhuskin A employed a directed ortho metalation approach as shown in Scheme 1(Neighbors, J. D.; et al., J Nat. Prod. 2008, 71, 1949). Lithiation of the ring may be directed by the MOM protecting group and presumably the benzylic alcohol anion of the known starting material (5) to afford the intermediate anion. Transmetalation to the copper species followed by treatment with prenyl bromide gave the final product alcohol (6) in modest yields as the only easily isolated product. In attempts to improve the yield use of copper iodide and TMEDA was explored because this had been previously shown with halogen metal exchange reactions in similar systems to afford superior yields (Topczewski, J. J.; et al., J. Org. Chem. 2011, 76, 909). The addition of TMEDA and use of copper iodide in ether afforded a mixture of the arene 6 and the isomeric prenylated compound 7 in a 1:1.2 ratio (Table 1, entry 1) and a combined yield of 36%.

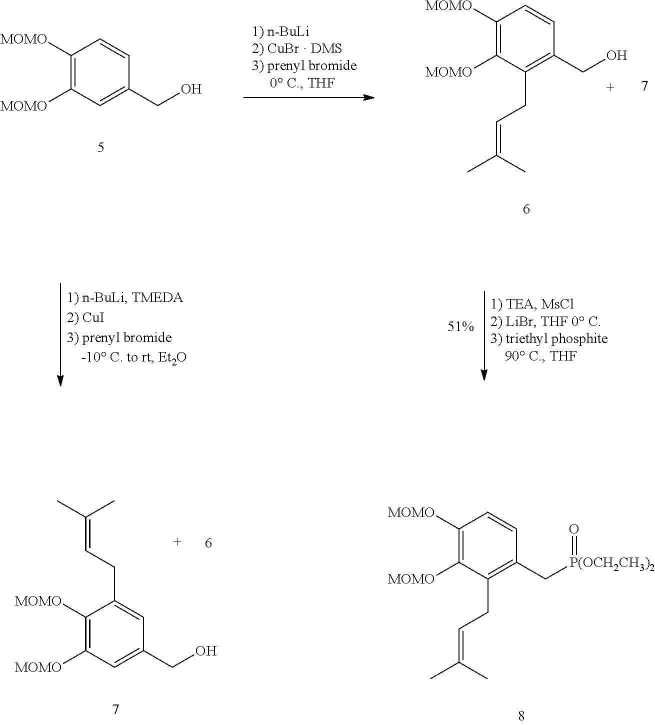

A more thorough exploration of the conditions showed that either regioisomer could be made with some selectivity. Slightly colder reaction temperatures afforded the best combined yield of products favoring compound 7 (entry 2). Forgoing the transmetalation step improved the ratio of compound 7 to 6 but the overall yield was particularly disappointing (entry 3). Reaction at room temperature in THF with copper bromide but without TMEDA afforded the alternate regioisomer 6 as the predominant product (6:7 2.9:1 entry 4) in a combined yield of 47%. Variation of the reaction temperature and the scale, which also might afford better control of the reaction temperature, did not improve this ratio (entries 5-7).

TABLE 1

| | | | | | Prenyl | | | | |
|---|---|---|---|---|---|---|---|---|---|
| | Scale | TMEDA | n-BuLi | CuBr•DMS | bromide | Solvent | | | |
| Trial | (mmol) | (mmol) | (mmol) | (mmol) | (mmol) | [conc.] | T | 6:7 | % Yield |
| 1 | 6.51 | 14.01 | 14.25 | 6.53^{a} | 7.16 | Et₂O [0.07M] | −10° C. to rt | 1.0:1.2 | 36 |
| 2 | 7.97 | 16.67 | 17.5 | 7.98^{a} | 11.93 | Et₂O [0.06M] | −20 to 0° C. to rt | 1.0:1.9 | 25 |
| 3 | 4.18 | 8.67 | 9.2 | NA | 6.31 | Et₂O [0.06M] | −20 to 0° C. to rt | 1.0:4.0 | 10 |
| 4 | 4.46 | NA | 9.52 | 4.91 | 4.94 | THF [0.13M] | rt | 2.9:1.0 | 47 |
| 5 | 0.92 | NA | 1.95 | 1.02 | 1.11 | THF [0.13M] | rt | 1.1:1.0 | 25 |
| 6 | 1.31 | NA | 2.75 | 1.11 | 1.53 | THF [0.13M] | 0° C. | 1.0:1.0 | 39 |
| 7 | 4.53 | NA | 9.52 | 4.98 | 4.94 | THF [0.13M] | 0° C. | 1.8:1.0 | 51 |

Effect of temperature and other parameters on directed ortho metalation of compound 5. ^{a} In these experiments, copper iodide was used.

With a viable route to compound 6 the preparation of the compound 4 followed. Treatment of the benzylic alcohol 6 with methanesulfonyl chloride in trimethylamine gave the mesylate which was converted into the bromide without isolation. An Arbuzov reaction was carried out by heating the bromide with triethyl phosphite to give the desired phosphonate 8 in moderate yield. Horner-Wadsworth-Emmons coupling of phosphonate 8 and the known aldehyde 9 (Hartung, A. M.; et al., J. Nat. Prod. 2014, 77, 311) afforded the protected stilbene 10 (Scheme 2). Global deprotection of the methoxymethyl ether groups by treatment with p-toluenesulfonic acid in methanol gave the desired compound 4.

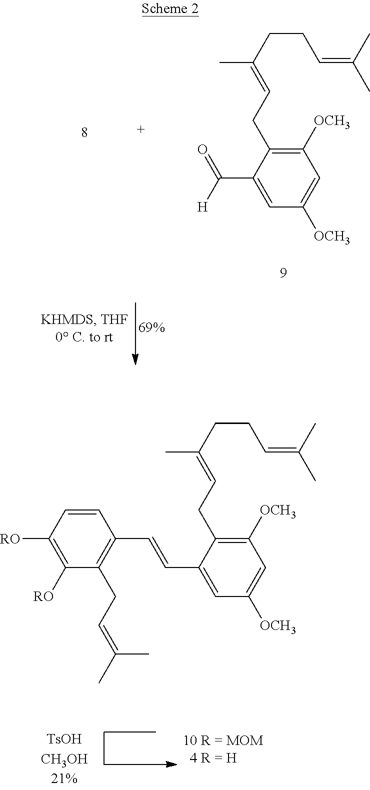

### Synthesis of Compounds 7, 10 and 4.

Compound 7. Alcohol 5 (719 mg, 3.2 mmol) in Et₂O (4 mL) was added dropwise to a solution of n-BuLi (2.4 M solution in hexanes, 2.9 mL, 7.0 mmol) and TMEDA (1.0 mL, 6.7 mmol) in Et₂O (50 mL) at −10° C. The mixture was allowed to stir for 15 min and then solid CuI (600 mg, 3.2 mmol) was added. Once the reaction turned a dark gray color (10 min) prenyl bromide (0.40 mL, 3.4 mmol) in Et₂O (5 mL) was added dropwise (˜7 min). The reaction was allowed to warm from −10° C. to rt and stir at rt for 17 h and then quenched by the addition of H₂O, extracted with Et₂O, dried (MgSO₄), and filtered, and the filtrate was concentrated in vacuo. Final purification by flash column chromatography (10% EtOAc in hexanes) afforded prenylated compound 7 (254 mg, 27%) as a light yellow oil: ¹H NMR (500 MHz, CDCl₃) δ 7.01 (d, J=2.0 Hz, 1H), 6.83 (d, J=1.8 Hz, 1H), 5.31-5.28 (m, 1H), 5.19 (s, 2H), 5.10 (s, 2H), 4.59 (d, J=3.4 Hz, 2H), 3.60 (s, 3H), 3.50 (s, 3H), 3.41 (d, J=7.3 Hz, 2H), 1.74 (s, 3H), 1.72 (s, 3H); ¹³C NMR (75 MHz, CDCl₃) δ 149.6, 143.8, 137.0, 136.0, 132.5, 122.5, 121.4, 112.6, 98.9, 94.9, 64.8, 57.3, 56.1, 28.4, 25.6, 17.7; HRMS (ESI) m/z calcd for C₁₆H₂₄O₅Na (M+H)⁺ 319.1521, found 319.1523.

Compound 10. To a solution of aldehyde 9 (26 mg, 0.09 mmol) and phosphonate 8 (63 mg, 0.15 mmol) in THF (1.7 mL) at 0° C. was added KHMDS (1.0 M solution in THF, 0.43 mL, 0.43 mmol). The reaction was allowed to warm to room temperature while stirring for 18 h. It was then quenched by the addition of NH4C1, and the resultant mixture was extracted with EtOAc. The combined organic extracts were washed with brine, dried (MgSO4), filtered, and concentrated in vacuo. Final purification by preparative TLC (30% EtOAc in hexanes) provided stilbene 10 (34 mg, 69%) as a yellow oil: ¹H NMR (500 MHz, CDCl₃) δ 7.30 (d, J=8.4 Hz, 1H), 7.12-7.11 (m, 2H), 7.02 (d, J=8.7 Hz, 1H), 6.70 (d, J=2.6 Hz, 1H), 6.42 (d, J=2.3 Hz, 1H), 5.21 (s, 2H), 5.18-5.12 (m, 1H), 5.12-5.10 (m, 3H), 5.07-5.04 (m, 1H), 3.83 (s, 3H), 3.81 (s, 3H), 3.60 (s, 3H), 3.56 (d, J=6.5 Hz, 2H), 3.51 (s, 3H), 3.42 (d, J=6.3 Hz, 2H), 2.07-2.02 (m, 2H), 1.98-1.95 (m, 2H), 1.80 (s, 3H), 1.78 (s, 3H), 1.68 (s, 3H), 1.61 (s, 3H), 1.55 (s, 3H); 13C NMR (125 MHz, CDCl₃) δ 158.5, 158.4, 149.3, 144.6, 138.3, 134.5, 134.3, 131.9, 131.4, 131.2, 128.0, 127.4, 124.3, 123.5, 123.3, 122.0, 121.2, 114.2, 101.6, 99.2, 98.0, 95.1, 57.6, 56.2, 55.7, 55.3, 39.7, 26.8, 25.8, 25.6, 25.6, 24.3, 18.2, 17.6, 16.3; HRMS (ESI) m/z calcd for C₃₅H₄₉O₆ (M+H)⁺ 565.3529, found 565.3541.

Compound 4. To a solution of the bis(methoxymethyl) ether 10 (33 mg, 0.06 mmol) in MeOH (5.8 mL) was added TsOH (46 mg, 0.24 mmol). The reaction was allowed to stir for 3 d, and was quenched by the addition of NaHCO₃. The resultant mixture was extracted with EtOAc, and the combined organic extracts were dried (MgSO₄), filtered, and concentrated in vacuo. Phenol 4 (6 mg, 21%) was obtained as a yellow oil after final purification by preparative TLC (30% EtOAc in hexanes): 1H NMR (500 MHz, CDCl₃) δ 7.14-7.06 (m, 3H), 6.77 (d, J=8.7 Hz, 1H), 6.71 (d, J=2.7 Hz, 1H), 6.42 (d, J=2.0 Hz, 1H), 5.44 (br s, 2H), 5.24-5.21 (m, 1H), 5.12-5.10 (m, 1H), 5.06-5.03 (m, 1H), 3.84 (s, 3H), 3.31 (s, 3H), 3.51 (d, J=6.5 Hz, 2H), 3.41 (d, J=7.0 Hz, 2H), 2.06-2.02 (m, 2H), 1.97-1.94 (m, 2H), 1.84 (s, 3H), 1.76-1.74 (m, 6H), 1.62 (s, 3H), 1.55 (s, 3H); 13C NMR (125 MHz, CDCl₃) δ 158.6, 158.5, 143.6, 142.0, 138.4, 134.6, 134.4, 131.3, 130.1, 128.2, 127.7, 125.9, 124.4, 123.5, 121.9, 121.2, 118.8, 113.2, 101.9, 97.9, 55.7, 55.3, 39.7, 26.8, 25.9, 25.7, 25.6, 24.4, 18.1, 17.6, 16.2; HRMS (ESI) m/z calcd for C₃₁H₄₁O₄ (M+H)⁺ 477.3005, found 477.3017.

### Biological Testing for Opioid Receptor Activity

All compounds were initially screened for intrinsic and antagonist activity at 10 μM in the [³⁵S]GTPγS binding assay at the human κ, and the μ, and δ opioid receptors over-expressed in CHO cells. These cell lines were kindly provided by Temple University (κ) and SRI International (μ and δ). Compounds identified as antagonist were characterized for functional antagonism (Ke) and selectivity by measuring the ability of the test compounds to inhibit stimulated [³⁵S]GTPγS binding produced by one of the selective agonists DAMGO (μ), DPDPE (δ), or U69,593 (κ). Agonist concentration-response curves were run in the presence or absence of a single concentration of test compound.

The test compounds were assayed in duplicate in 1.4-mL polypropylene tubes in a 96-well format. CHO membrane homogenates (20-40 pg protein) were incubated with a positive control or the test compound, 0.1 nM [³⁵S]GTP-γ-S and 1 μM GDP in 50 mM HEPES buffer (pH 7.4) at room temperature for 1 h, after which bound radioligand was separated from free radioligand via rapid vacuum filtration over GF-B filters with a Brandel Scientific (Gaithersburg, Md.) 96-well harvester. Bound radioactivity is determined using a TopCount 12-detector instrument (Packard Instruments) using standard scintillation counting techniques. Bound radioactivity is normalized to samples containing vehicle (basal binding). A 4-parameter logistic function was fit to these data to calculate the EC₅₀ and Emax values using Prism (v. 6; Graph Pad Software, San Diego, Calif.). The Ke values were calculated using the formula Ke=[L]/DR−1, where [L] is the concentration of test compound, and DR is the ratio of agonist EC₅₀ value in the presence or absence of test compound.

### Docking Studies

The 3D ligand structures were built in ArgusLab 4.0.1 and were saved as mol files after energy minimization using the PM3 level of theory. The mol file was used as ligand for AutoDock Vina with the Protein Data Bank file 4EJ4 (delta opioid receptor bound to naltrindole antagonist) used as the receptor. The receptor was prepared by removing the bound naltrindole ligand, removing solvent molecules, merging non-polar hydrogens and adding Gastegier charges using AutoDock tools. Two docking runs were run for each ligand using a boxed region of the entire receptor and limiting the region of interest to the ligand binding site to assure no alternative binding sites were accessible. These both gave the same results. The conformation with the lowest score was used for further analysis. All images were rendered using the Chimera suite of molecular visualization tools.

### Discussion

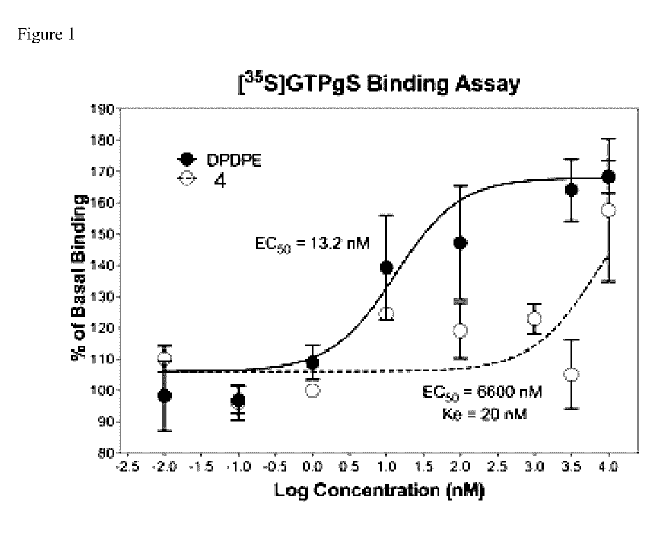

Compound 4 was tested for opioid receptor activity by first assessing if intrinsic agonist activity was present. After finding no agonist activity, this compound was tested for antagonist selectivity against the mu, delta and kappa opioid receptors (MOP, DOP, and KOP). To our surprise compound 4 displayed strong antagonist activity that was very selective for the DOP (Kₑ=25 nM, KOP/DOP>400, MOP/DOP>400, FIG. 1). This was an intriguing result. In essence, moving the prenyl substituent from a position ortho to the stilbene junction in isomer 4 to a position meta to the central olefin, as in compound 3 (Hartung, A. M.; et al., J. Nat. Prod. 2014, 77, 311), shifted the activity from highly delta selective to highly kappa selective (Table 2).

TABLE 2

| | apparent affinity (Kₑ) | | |
|---|---|---|---|
| | of antagonists in μM | | |
| Compound | DOP | KOP | MOP |
| 1 | 2.9 | 0.2 | 570 |
| 3 | >10 | 0.15 | >10 |
| 4 | 0.025 | >10 | >10 |

Apparent affinities of pawhuskin A (1), compound 3 and compound 4.

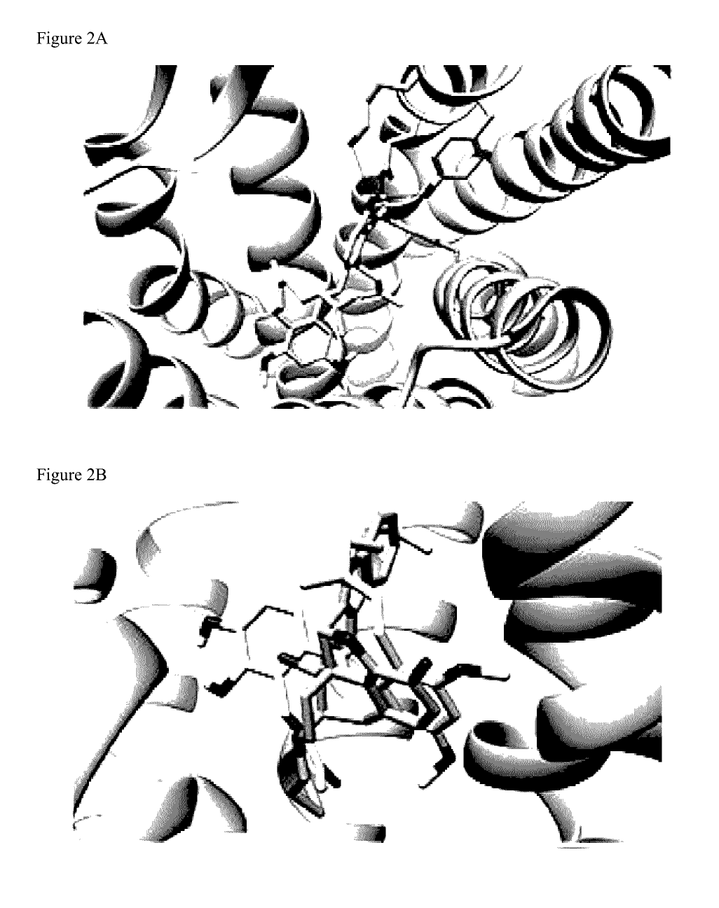

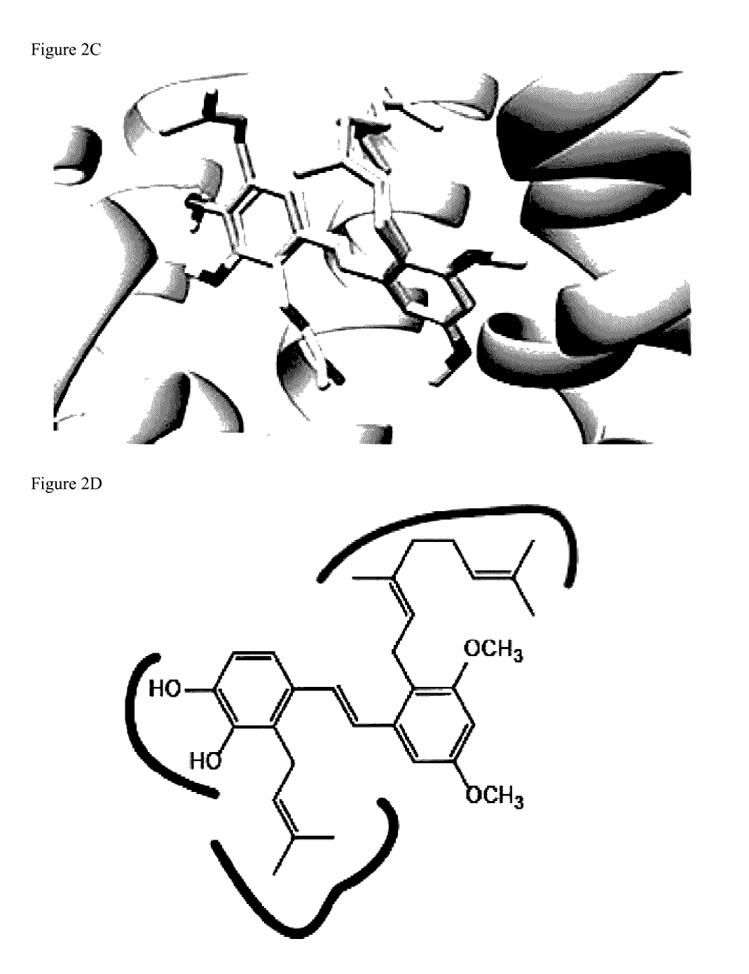

In order to rationalize this dramatic change in selectivity when the prenyl group is shifted, docking studies were conducted. The structure of the mouse DOP with the bound antagonist naltrindole was solved in 2012 by the Kobilka group (Granier, S.; et al., Nature 2012, 485, 400). This structure was used with the Autodock Vina (Trott, O.; Olson, A. J. Journal of Computational Chemistry 2010, 31, 455) software package to perform docking of compounds 3 and 4 into the receptor. Compound 4 fits neatly into the DOP receptor binding pocket with the free phenols of the catechol ring predicted to make hydrogen bonds with LYS108, GLN105, TYR109, and TYR308 of the DOP structure (FIG. 2A, visualization was conducted using the Chimera software suite; Granier, S.; et al., Nature 2012, 485, 400, Pettersen, E. F.; et al., Journal of Computational Chemistry 2004, 25, 1605). Interestingly, the hydrophobic isoprenoid groups of compound 4 overlap quite well with the indole (geranyl group) and cyclopropylmethyl (prenyl group) groups of naltrindole (FIG. 2B). When the KOP selective compound 3 is docked using the same procedure, the lowest energy conformation overlaps almost perfectly with that of the predicted lowest energy conformation of compound 4. The prominent exception is the prenyl group which is now directed up and away from the region occupied by the cyclopropylmethyl moiety of naltrindole in the x-ray structure (FIG. 2C). This change demonstrates that compound 3 lacks key hydrophobic interactions that presumably support the binding of naltrindole and compound 4 to the DOP. The lowest energy docking pose for compound 4 has a score of −8.9 vs. a −7.3 for the lowest energy pose of compound 3. This correlates nicely with the large difference in the functional binding assay.

The overall binding motif of compound 4 can be viewed in the context of the message and address concept of opioid binding in which the geranyl group, like the indole of naltrindole, extends into a region of the receptor that confers selectivity. In contrast the prenyl group and the phenols are the message which allows binding to key parts of the receptor architecture, in this case blocking the ability of ligand to bind and signal (FIG. 2D). In contrast, for the docked pose of kappa selective compound 3 the hydrophobic contributions to the message part of the binding are not possible. This dramatically reduces the overall interaction as depicted by the docking score.

The differences with respect to the docking of the natural product Pawhuskin A (1, FIG. 2C) are more difficult to rationalize. If one assumes that Pawhuskin A adopts an orientation similar to the delta selective compound 4, it leads to an intermediate docking score of −8.4. In this orientation, the phenols of Pawhuskin A are orientated away from the space occupied by the methoxy groups of compound 4. If that orientation improves the hydrophobic interaction between compound 4 and the receptor, it would lead to stronger binding. A favorable orientation of the prenyl group of Pawhuskin A may compensate for some of the reduction caused by the absence of the methoxy groups, and allow functional antagonism at the DOP albeit with reduced apparent affinity.

Thus, a highly selective delta opioid receptor antagonist (compound 4) based on the stilbene motif of the pawhuskin natural products has been discovered. Studies on directed ortho metalation of compound 5 have uncovered conditions which favor prenylation ortho to the benzylic position (i.e., compound 6) or meta to this substituent (compound 7). Incorporation of compound 7 into the final stilbene has yielded the kappa-selective compound 3, while incorporation of the isomeric 6 has provided compound 4 which is delta selective. Docking studies have shed some light on the potential differences in the binding modes of the stilbene isomers 3 and 4 to the DOP, and provided some rationale for the large difference in selectivity.

### EXAMPLE 2

Preparation of compounds 11, 12, 13 and 14.

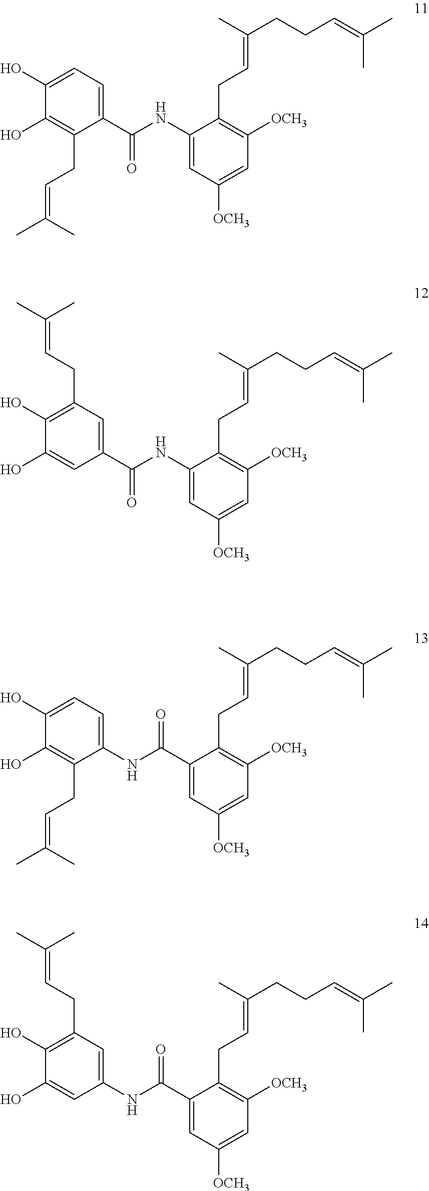 [SMILES: COc1cc(NC(=O)c2ccc(O)c(O)c2CC=C(C)C)c(C/C=C(\C)CCC=C(C)C)c(OC)c1 ; COc1cc(NC(=O)c2cc(O)c(O)c(CC=C(C)C)c2)c(C/C=C(\C)CCC=C(C)C)c(OC)c1 ; COc1cc(OC)c(C/C=C(\C)CCC=C(C)C)c(C(=O)Nc2ccc(O)c(O)c2CC=C(C)C)c1 ; COc1cc(OC)c(C/C=C(\C)CCC=C(C)C)c(C(=O)Nc2cc(O)c(O)c(CC=C(C)C)c2)c1]

Compounds 11, 12, 13 and 14 were prepared as outlined in Schemes 3 and 4.

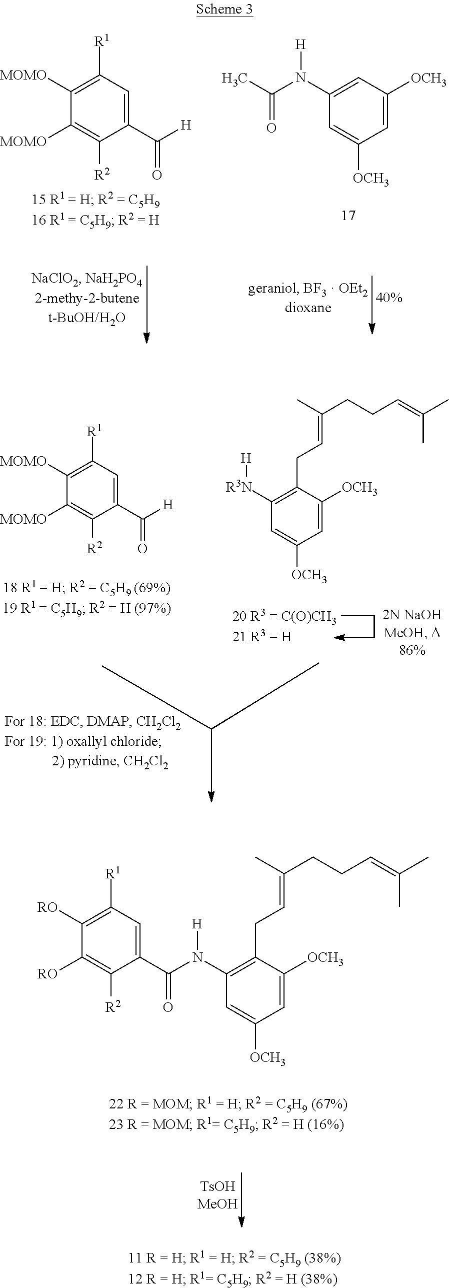

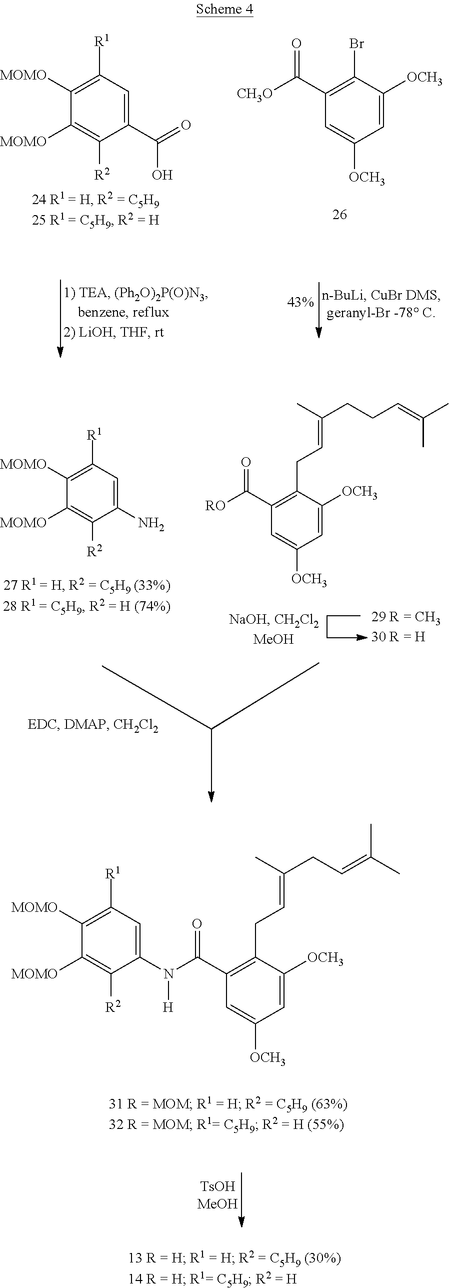

### Experimental Data

### Preparation of (E)-N-(2-(3,7-Dimethylocta-2,6-dienyl)-3,5-dimethoxyphenyl)-3,4-dihydroxy-2-(3-methylbut-2-enyl)benzamide (11)

To a stirred solution of the MOM acetal 22 (25 mg, 43 μmol) in MeOH (13 mL) was added p-TsOH.H₂O (0.031 g, 0.16 mmol). The flask was sealed and stirred overnight at room temperature. The solution was diluted with EtOAc and then washed with saturated aqueous NaHCO₃. The layers were separated and the aqueous layer was extracted with EtOAc (3×). The combined organics were washed with brine, dried over Na₂SO₄ and filtered, and the filtrate was concentrated in vacuo. Final purification using an ISCO Combiflash Rf chromatography gradient (20-460% EtOAc in hexanes) afforded compound 11 (8 mg, 38%) as an off-white solid: ¹H NMR (400 MHz, CDCl₃) δ 7.74 (s, 1H), 7.47 (s, 1H), 6.89 (d, J=8.2 Hz, 1H), 6.73 (d, J=8.2 Hz, 1H), 6.32 (d, J=2.3 Hz, 1H), 5.31-5.23 (m, 1H), 5.08-4.98 (m, 2H), 3.82 (s, 3H), 3.80 (s, 3H), 3.60 (d, J=6.7 Hz, 2H), 3.31 (d, J=6.7 Hz, 2H), 1.99-1.85 (m, 4H), 1.75 (s, 3H), 1.73 (s, 3H), 1.64 (s, 3H), 1.54 (s, 3H), 1.46 (s, 3H).

### Preparation of (E)-N-(3,4-Dihydroxy-2-(3-methylbut-2-enyl)phenyl)-2-(3,7-dimethylocta-2,6-dienyl)-3,5-dimethoxybenzamide (13)

To a stirred solution of the MOM acetal 31 (28 mg, 48 μmol) in MeOH (4.8 mL) was added p-TsOH.H₂O (35 mg, 0.18 mmol). The flask was sealed and stirred overnight at room temperature. The solution was diluted with EtOAc and then washed with saturated aqueous NaHCO₃. The layers were separated and the aqueous layer was extracted with EtOAc (3×). The combined organic extracts were washed with brine, dried over Na₂SO₄, and filtered, and the filtrate was concentrated in vacuo. Final purification using an ISCO Combiflash Rf chromatography gradient (0-40% EtOAc in hexanes) afforded compound 13 (7 mg, 30%) as an off-white solid: ¹H NMR (400 MHz, CDCl₃) δ 7.46 (s, 1H), 6.97 (d, J=8.5 Hz, 1H), 6.64 (d, J=2.4 Hz, 1H), 6.58 (d, J=8.6 Hz, 1H), 6.56 (d, J=2.4 Hz, 1H), 5.17 (t, J=6.1 Hz, 1H), 5.12 (t, J=7.0 Hz, 1H), 5.03 (t, J=6.7 Hz, 1H), 3.84 (s, 3H), 3.82 (s, 3H), 3.50 (d, J=6.7 Hz, 2H), 3.33 (d, J=6.6 Hz, 2H), 2.02-1.96 (m, 2H), 1.96-1.90 (m, 2H), 1.63 (s, 6H), 1.63 (s, 3H), 1.55 (s, 3H), 1.54 (s, 3H); ¹³C NMR (101 MHz, CDCl₃) δ 169.4, 159.0, 158.9, 143.0, 142.3, 137.7, 136.0, 134.0, 131.4, 128.0, 124.2, 123.1, 122.1, 121.6, 120.8, 117.0, 113.4, 103.1, 100.6, 55.8, 55.5, 39.8, 29.7, 26.8, 25.6 (s, 2C), 25.5, 24.7, 17.7, 16.3; HRMS (TOF MS ES+) m/z calculated for C₃₀H₄₀NO₅(M+H)⁺ 494.2906, found 494.2905.

All publications, patents, and patent documents cited herein are incorporated by reference herein, as though individually incorporated by reference. The invention has been described with reference to various specific and preferred embodiments and techniques. However, it should be understood that many variations and modifications may be made while remaining within the spirit and scope of the invention.

What is claimed is:

1. A compound of formula I: 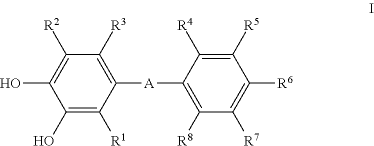
wherein;
A is 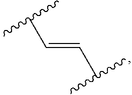 [SMILES: */C=C/*]
—C(═O)NH— or —NHC(═O)—;
R¹ is a C₁-C₁₀ saturated or unsaturated hydrocarbon chain, aryl, heteroaryl, aryl(C₁-C₆)alkyl- or heteroaryl(C₁-C₆)alkyl- wherein any C₁-C₁₀ saturated or unsaturated hydrocarbon chain of R¹ is optionally substituted with one or more groups independently selected from halogen and —O(C₁-C₆)alkyl and any aryl, heteroaryl, aryl(C₁-C₆)alkyl- or heteroaryl(C₁-C₆)alkyl- of R¹ is optionally substituted with one more groups independently selected from halogen, (C₁-C₆)alkyl and —O(C₁-C₆)alkyl, and R² is hydrogen, halogen or —OH; or
R¹ is hydrogen, halogen or —OH, and R² is a C₁-C₁₀ saturated or unsaturated hydrocarbon chain, aryl, heteroaryl, aryl(C₁-C₆)alkyl- or heteroaryl(C₁-C₆)alkyl- wherein any C₁-C₁₀ saturated or unsaturated hydrocarbon chain of R² is optionally substituted with one or more groups independently selected from halogen and —O(C₁-C₆)alkyl and any aryl, heteroaryl, aryl(C₁-C₆)alkyl- or heteroaryl(C₁-C₆)alkyl- of R² is optionally substituted with one more groups independently selected from halogen, (C₁-C₆)alkyl and —O(C₁-C₆)alkyl, and R² is hydrogen, halogen or —OH;
R³ is hydrogen or halogen;
R⁴ is a C₃-C₁₄ saturated or unsaturated hydrocarbon chain, aryl, heteroaryl, aryl(C₁-C₆)alkyl- or heteroaryl(C₁-C₆)alkyl- wherein any C₃-C₁₄ saturated or unsaturated hydrocarbon chain of R⁴ is optionally substituted with one or more groups independently selected from halogen and —O(C₁-C₆)alkyl and any aryl, heteroaryl, aryl(C₁-C₆)alkyl- or heteroaryl(C₁-C₆)alkyl- of R⁴ is optionally substituted with one more groups independently selected from halogen, (C₁-C₆)alkyl and —O(C₁-C₆)alkyl;
R⁵ is (C₁-C₄)alkyl, (C₂-C₄)alkenyl, (C₂-C₄)alkynyl or —OR^{a} wherein any (C₁-C₄)alkyl, (C₂-C₄)alkenyl or (C₂-C₄)alkynyl of R⁵ is optionally substituted with one or more halogen;
R⁶ is hydrogen, halogen, (C₁-C₄)alkyl, (C₂-C₄)alkenyl, (C₂-C₄)alkynyl or —OR^{b} wherein any (C₁-C₄)alkyl, (C₂-C₄)alkenyl or (C₂-C₄)alkynyl of R⁵ is optionally substituted with one or more halogen;
R⁷ is (C₁-C₄)alkyl, (C₂-C₄)alkenyl, (C₂-C₄)alkynyl or —OR^{c} wherein any (C₁-C₄)alkyl, (C₂-C₄)alkenyl or (C₂-C₄)alkynyl of R⁵ is optionally substituted with one or more halogen;
R⁸ is hydrogen or halogen;
R^{a} is (C₁-C₄)alkyl, (C₂-C₄)alkenyl or (C₂-C₄)alkynyl, wherein any (C₁-C₄)alkyl, (C₂-C₄)alkenyl or (C₂-C₄)alkynyl of R^{a} is optionally substituted with one or more halogen;
R^{b} is (C₁-C₄)alkyl, (C₂-C₄)alkynyl or (C₂-C₄)alkynyl, wherein any (C₁-C₄)alkyl, (C₂-C₄)alkenyl or (C₂-C₄)alkynyl of R^{b} is optionally substituted with one or more halogen; and
R^{c} is a (C₁-C₄)alkyl, (C₂-C₄)alkenyl or (C₂-C₄)alkynyl, wherein any (C₁-C₄)alkyl, (C₂-C₄)alkenyl or (C₂-C₄)alkynyl of R^{c} is optionally substituted with one or more halogen;
or a salt thereof;
provided the compound is not 5-((E)-2-((E)-3,7-dimethylocta-2,6-dien-1-yl)-3,5-dimethoxystyryl)-3-(3-methylbut-2-en-1-yl)benzene-1,2-diol or a salt thereof.

2. The compound of claim 1 which is a compound of formula I″: 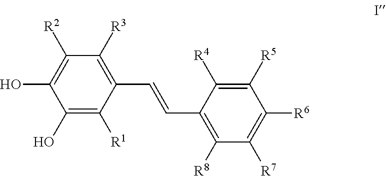
wherein;
R¹ is a C₁-C₁₀ saturated or unsaturated hydrocarbon chain, aryl, heteroaryl, aryl(C₁-C₆)alkyl- or heteroaryl(C₁-C₆)alkyl- wherein any C₁-C₁₀ saturated or unsaturated hydrocarbon chain of R¹ is optionally substituted with one or more groups independently selected from halogen and —O(C₁-C₆)alkyl and any aryl, heteroaryl, aryl(C₁-C₆)alkyl- or heteroaryl(C₁-C₆)alkyl- of R¹ is optionally substituted with one more groups independently selected from halogen, (C₁-C₆)alkyl and —O(C₁-C₆)alkyl;
R² is hydrogen, halogen or —OH;
R³ is hydrogen or halogen;
R⁴ is a C₃-C₁₄ saturated or unsaturated hydrocarbon chain, aryl, heteroaryl, aryl(C₁-C₆)alkyl- or heteroaryl(C₁-C₆)alkyl- wherein any C₃-C₁₄ saturated or unsaturated hydrocarbon chain of R⁴ is optionally substituted with one or more groups independently selected from halogen and —O(C₁-C₆)alkyl and any aryl, heteroaryl, aryl(C₁-C₆)alkyl- or heteroaryl(C₁-C₆)alkyl- of R⁴ is optionally substituted with one more groups independently selected from halogen, (C₁-C₆)alkyl and —O(C₁-C₆)alkyl;
R⁵ is (C₁-C₄)alkyl, (C₂-C₄)alkenyl, (C₂-C₄)alkynyl or —OR^{a} wherein any (C₁-C₄)alkyl, (C₂-C₄)alkenyl or (C₂-C₄)alkynyl of R⁵ is optionally substituted with one or more halogen;
R⁶ is hydrogen, halogen, (C₁-C₄)alkyl, (C₂-C₄)alkenyl, (C₂-C₄)alkynyl or —OR^{b} wherein any (C₁-C₄)alkyl, (C₂-C₄)alkenyl or (C₂-C₄)alkynyl of R⁵ is optionally substituted with one or more halogen;
R⁷ is (C₁-C₄)alkyl, (C₂-C₄)alkenyl, (C₂-C₄)alkynyl or —OR^{c} wherein any (C₁-C₄)alkyl, (C₂-C₄)alkenyl or (C₂-C₄)alkynyl of R⁵ is optionally substituted with one or more halogen;
R⁸ is hydrogen or halogen;
R^{a} is (C₁-C₄)alkyl, (C₂-C₄)alkenyl or (C₂-C₄)alkynyl, wherein any (C₁-C₄)alkyl, (C₂-C₄)alkenyl or (C₂-C₄)alkynyl of R^{a} is optionally substituted with one or more halogen;
R^{b} is (C₁-C₄)alkyl, (C₂-C₄)alkenyl or (C₂-C₄)alkynyl, wherein any (C₁-C₄)alkyl, (C₂-C₄)alkenyl or (C₂-C₄)alkynyl of R^{b} is optionally substituted with one or more halogen; and
R^{c} is a (C₁-C₄)alkyl, (C₂-C₄)alkenyl or (C₂-C₄)alkynyl, wherein any (C₁-C₄)alkyl, (C₂-C₄)alkenyl or (C₂-C₄)alkynyl of R^{c} is optionally substituted with one or more halogen;
or a salt thereof.

3. The compound of claim 1, wherein R³ is hydrogen.

4. The compound of claim 1, wherein R⁶ is hydrogen.

5. The compound of claim 1, wherein R⁸ is hydrogen.

6. The compound of claim 1, which is a compound of formula Ia: 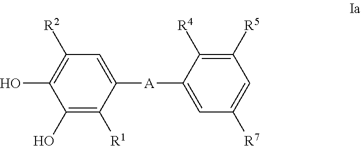
or a salt thereof.

7. The compound of claim 1, wherein R⁵ is —OR^{a}.

8. The compound of claim 1, wherein R⁷ is —OR^{c}.

9. The compound of claim 1, which is a compound of formula Ib: 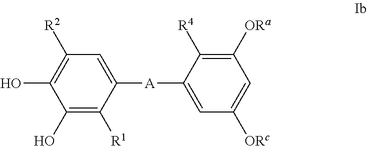
or a salt thereof.

10. The compound of claim 1, wherein R¹ is a C₃-C₇ saturated or unsaturated hydrocarbon chain wherein the hydrocarbon chain is optionally substituted with one or more groups independently selected from halogen and —O(C₁-C₆)alkyl and R² is hydrogen, halogen or —OH.

11. The compound of claim 1, wherein R² is a C₃-C₇ saturated or unsaturated hydrocarbon chain wherein the hydrocarbon chain is optionally substituted with one or more groups independently selected from halogen and —O(C₁-C₆)alkyl and R¹ is hydrogen, halogen or —OH.

12. The compound of claim 1, wherein R¹ is a C₃-C₇ saturated or unsaturated hydrocarbon chain wherein the hydrocarbon chain is optionally substituted with one or more groups independently selected from halogen and —O(C₁-C₆)alkyl, R² is hydrogen, halogen or —OH, and A is

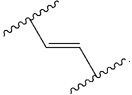 [SMILES: */C=C/*]

13. The compound of claim 1, wherein A is —C(═O)NH— or —NHC(═O)—, and R¹ is a C₃-C₇ saturated or unsaturated hydrocarbon chain wherein the hydrocarbon chain is optionally substituted with one or more groups independently selected from halogen and —O(C₁-C₆)alkyl and R² is hydrogen, halogen or —OH; or A is —C(═O)NH— or —NHC(═O)—, and R² is a C₃-C₇ saturated or unsaturated hydrocarbon chain wherein the hydrocarbon chain is optionally substituted with one or more groups independently selected from halogen and —O(C₁-C₆)alkyl and R¹ is hydrogen, halogen or —OH.

14. The compound of claim 1, which is a compound of formula Ic, 1d, or 1e: 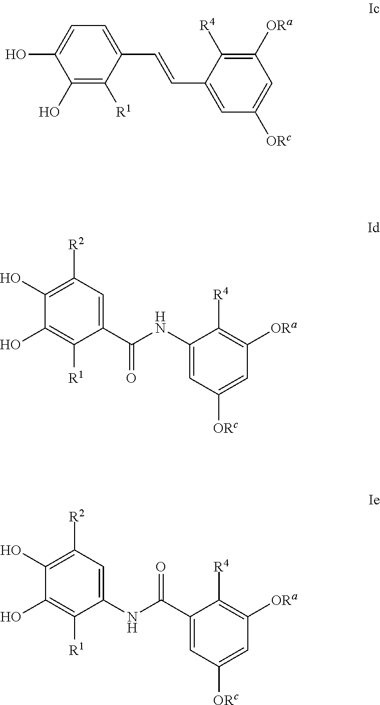
or a salt thereof.

15. The compound of claim 1, wherein R⁴ is a C₈-C₁₂ saturated or unsaturated hydrocarbon chain wherein the hydrocarbon chain is optionally substituted with one or more groups independently selected from halogen and —O(C₁-C₆)alkyl.

16. The compound of claim 1, wherein R⁴ is:

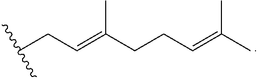 [SMILES: *C/C=C(\C)CCC=C(C)C]

17. The compound of claim 1, wherein R^{a} and R^{c} are each independently (C₁-C₄)alkyl wherein any (C₁-C₄)alkyl of R^{a} or R^{c} is optionally substituted with one or more halogen.

18. The compound of claim 1 that is: 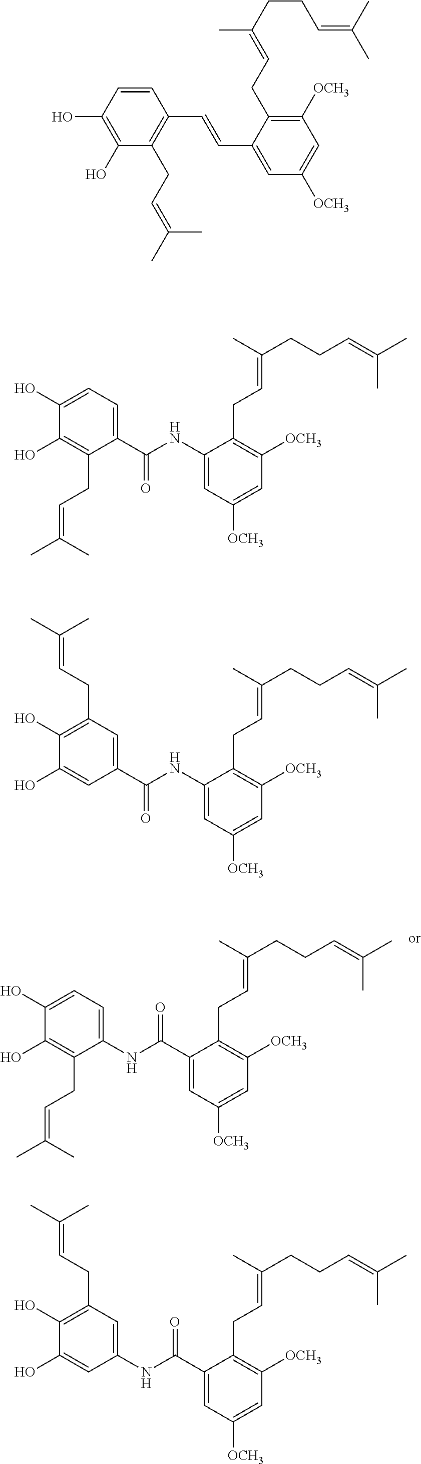 [SMILES: COc1cc(/C=C/c2ccc(O)c(O)c2CC=C(C)C)c(C/C=C(\C)CCC=C(C)C)c(OC)c1 ; COc1cc(NC(=O)c2ccc(O)c(O)c2CC=C(C)C)c(C/C=C(\C)CCC=C(C)C)c(OC)c1 ; COc1cc(NC(=O)c2cc(O)c(O)c(CC=C(C)C)c2)c(C/C=C(\C)CCC=C(C)C)c(OC)c1 ; COc1cc(OC)c(C/C=C(\C)CCC=C(C)C)c(C(=O)Nc2ccc(O)c(O)c2CC=C(C)C)c1 ; COc1cc(OC)c(C/C=C(\C)CCC=C(C)C)c(C(=O)Nc2cc(O)c(O)c(CC=C(C)C)c2)c1]
or a salt thereof.

19. A pharmaceutical composition comprising a compound of claim 1, or a pharmaceutically acceptable salt thereof, and a pharmaceutically acceptable carrier.

20. A method for treating pain, a psychiatric condition, or substance dependency in an animal, comprising administering to the animal in need thereof a therapeutically effective amount of a compound of claim 1, or a pharmaceutically acceptable salt thereof.
